(19) United States
(12) Patent Application Publication
(10) Pub. No.: US 2024/0091407 A1
(43) Pub. Date: Mar. 21, 2024

(54) CARTILAGE TISSUE ENGINEERING COMPLEX AND USE THEREOF

(71) Applicant: SHANGHAI RESTHETIC BIO CO., LTD, Shanghai (CN)

(72) Inventors: Guangdong ZHOU, Shanghai (CN); Yu LIU, Shanghai (CN); Zheng CI, Shanghai (CN)

(21) Appl. No.: 18/262,232

(22) PCT Filed: Jan. 17, 2022
(86) PCT No.: PCT/CN2022/072395

§ 371 Date: Jul. 20, 2023

(30) Foreign Application Priority Data
Jan. 20, 2021 (CN) 202110075619.9

(51) Int. Cl. A61L 27/36; A61L 27/20; A61L 27/38; A61L 27/52; A61L 27/54; A61L 27/56; C12N 5/00; C12N 5/077

(52) U.S. Cl. CPC A61L 27/3612; A61L 27/20; A61L 27/3608; A61L 27/3817; A61L 27/52; A61L 27/54; A61L 27/56; C12N 5/0018; C12N 5/0655; A61L 2430/06; C12N 2500/32; C12N 2500/38; C12N 2500/90; C12N 2501/15; C12N 2501/39

(57) ABSTRACT

A cartilage tissue engineering complex, e.g., a graft, has a cartilage gel or perichondral particles containing chondrocytes as well as a porous frame structure. The cartilage gel or perichondral particles is/are loaded on the porous frame structure to form a cartilage gel/perichondral particle-frame structure complex. The method for preparing the cartilage tissue engineering graft and a use of said graft in repair and reconstruction of articular cartilage are also provided.

### TECHNICAL FIELD

The present invention relates to the field of biomedical tissue engineering, in particular to a cartilage tissue engineering complex and preparation method thereof, and its use in joint repair.

### BACKGROUND

In recent years, with the rapid development of economy and society, various types of articular cartilage lesions such as various sports injuries, accidental injuries, and joint degenerative diseases have become more and more common, and cartilage lesions are difficult to repair depending on the patient's self-ability, so there is a huge clinical demand for joint repair. At present, the clinical treatment methods for articular cartilage lesions are mainly palliative treatment and restorative treatment. Palliative treatment mainly includes arthroscopic debridement and chondroplasty. This type of treatment can clean the uneven cartilage surface of the joint surface and remove cartilage fragments, etc., to restore the smooth and flat joint surface. This method is less traumatic and can relieve the patient's symptoms to a certain extent, but its curative effect is limited and cannot effectively relieve the development of arthritis. Restorative treatment includes microfracture treatment, osteochondral transplantation, etc. Although this kind of treatment can repair focal articular cartilage defects to a certain extent, the larger trauma can easily lead to complications in the donor area. In general, traditional treatment methods have poor curative effect. Autotransplantation lacks donors, and long-term complications of artificial joints are difficult to avoid.

In recent years, with the progress of tissue engineering, people have gradually begun to study the use of scaffolds or tissues constructed by tissue engineering to try to repair cartilage defects. Tissue engineering is an interdisciplinary subject involving cell biology, material science, engineering and bioreactor. It uses the basic principles and methods of life science and engineering to build the tissues needed by the human body to repair and replace the tissues or organs that have no function due to trauma and disease.

The chondrocyte-material complex constructed by the existing tissue engineering repair technology (ACI or MACI) has been gradually developed in clinical treatment, but there are still many disadvantages that are difficult to avoid: 1. The source of the cells is articular cartilage. Obtaining cells from the damaged joints will inevitably increase the damage of the original joints. 2. The proliferation ability of articular chondrocytes is limited. The construction cycle of early cell-material complex is long, and patients need to wait for a long time. 3. The strength of the cell-material complex is low, which is far less than the mechanical strength level of natural cartilage.

Therefore, new methods of joint repair are urgently needed.

### SUMMARY OF THE INVENTION

The purpose of the present invention is to provide a cartilage tissue engineering complex, a preparation method thereof, and its use in joint repair.

The first aspect of the present invention provides a cartilage tissue engineering complex, which comprises:
(a) a carrier comprising a porous frame structure; and
(b) cartilage gel or cartilage sheet pieces that are inoculated on or loaded on the carrier.

In another preferred embodiment, the complex comprises a complex formed by inoculating the cartilage gel or cartilage sheet pieces on the carrier and undergoing chondrogenic culture (in which the chondrocytes are loaded on the carrier and form a more closely integrated structure with the carrier).

In another preferred embodiment, the complex comprises a complex formed by inoculating the cartilage gel or cartilage sheet pieces on the carrier without chondrogenic culture.

In another preferred embodiment, the chondrocytes are derived from a human or non-human mammal.

In another preferred embodiment, the chondrocytes are derived from autologous chondrocytes or xenogenous chondrocytes, preferably autologous chondrocytes.

In another preferred embodiment, the chondrocytes are derived from elastic cartilage, fibrocartilage or hyaline cartilage.

In another preferred embodiment, the chondrocytes are taken from the subject's autologous chondrocytes.

In another preferred embodiment, the autologous chondrocytes are derived from elastic cartilage, fibrocartilage or hyaline cartilage.

In another preferred embodiment, the subject is a human or a non-human mammal.

In another preferred embodiment, the subject has a joint defect.

In another preferred embodiment, the joint defect is an articular cartilage defect.

In another preferred embodiment, the joint defect is a knee joint defect, an elbow joint defect, a hip joint defect, an ankle joint defect, a wrist joint defect, a mandibular joint defect, and a combination thereof.

In another preferred embodiment, the cartilage gel comprises a cell population of chondrocytes and extracellular matrix secreted by chondrocytes, wherein the extracellular matrix encapsulates the cell population, and the cartilage gel is in a gel state, and the density of chondrocytes is at least 1.0×10⁸ cells/ml or 1.0×10⁸ cells/g.

In another preferred embodiment, the cartilage gel is prepared by gelation culture of chondrocytes.

In another preferred embodiment, the gelation culture is an in vitro culture with gelation medium.

In another preferred embodiment, the gelation medium contains the following components: high-glucose DMEM medium containing 4 to 5 wt % glucose, 10% FBS (v/v) and 100 U/ml penicillin-streptomycin.

In another preferred embodiment, the adhesion rate of the cartilage gel is ≥90%, preferably ≥95%.

In another preferred embodiment, in the cartilage gel, the concentration of chondrocytes is 1.0×10⁸ cells/ml-10×10⁸ cells/ml, preferably 1.5-5×10⁸ cells/ml.

In another preferred embodiment, the cartilage gel is obtained by gelation culture for 2.5-5.5 days, preferably for 3-5 days.

In another preferred embodiment, the chondrocytes are derived from a human or non-human mammal.

In another preferred embodiment, the chondrocytes are derived from autologous chondrocytes or xenogenousa chondrocytes, preferably autologous chondrocytes.

In another preferred embodiment, the chondrocytes are taken from the subject's autologous chondrocytes.

In another preferred embodiment, the subject is a human or a non-human mammal.

In another preferred embodiment, the subject has a joint defect.

In another preferred embodiment, the joint defect is an articular cartilage defect.

In another preferred embodiment, the joint defect is a knee joint defect, an elbow joint defect, a hip joint defect, an ankle joint defect, a wrist joint defect, a mandibular joint defect, and a combination thereof.

In another preferred embodiment, the cartilage sheet pieces comprise a cell population composed of chondrocytes and extracellular matrix secreted by chondrocytes, wherein the extracellular matrix encapsulates the cell population, and the cartilage pieces are prepared by mincing the cartilage sheet, wherein the density of chondrocytes is at least 1.0×10⁸ cells/ml or 1.0×10⁸ cells/g.

In another preferred embodiment, the concentration of chondrocytes in the cartilage sheet is 1.0×10⁸ cells/ml-10×10⁸ cells/ml, preferably 1.5-5×10⁸ cells/ml.

In another preferred embodiment, the cartilage sheet is obtained by gelation culture for 6-30 days, preferably 7-20 days, and most preferably 10-15 days.

In another preferred embodiment, the gelation culture is an in vitro culture with gelation medium.

In another preferred embodiment, the gelation medium contains the following components: high-glucose DMEM medium containing 4 to 5 wt % glucose, 10% FBS (v/v) and 100 U/ml penicillin-streptomycin.

In another preferred embodiment, the thickness of the cartilage sheet is 0.2-0.25 mm.

In another preferred embodiment, the average volume of the cartilage sheet pieces is 0.2 μl.

In another preferred embodiment, the surface area of the cartilage sheet pieces is 0.05-10 mm², preferably, 1-5 mm², more preferably, the average area is 1 mm².

In another preferred embodiment, the porous frame structure is made of a biodegradable material selected from the group consisting of PCL, PGA, allogeneic bone repair material, xenogeneic bone repair material, or decalcified bone matrix.

In another preferred embodiment, the frame structure may further be loaded with gelatin, collagen, silk fibroin, hydrogel, and a combination thereof.

In another preferred embodiment, the frame structure is a decalcified bone matrix.

In another preferred embodiment, the decalcified bone matrix is derived from an allogeneic bone repair material.

In another preferred embodiment, the decalcified bone matrix is derived from a xenogeneic bone repair material.

In another preferred embodiment, the shape of the decalcified bone matrix comprises a cylinder, a cuboid or other specific shape.

In another preferred embodiment, the decalcified bone matrix has a thickness of 0.3-0.8 cm, preferably 0.4-0.6 cm, and most preferably 0.5 cm.

In another preferred embodiment, the decalcification amount of the decalcified bone matrix is 30% to 50%.

In another preferred embodiment, the cartilage gel/cartilage sheet pieces-frame structure complex can be converted into articular cartilage under the joint microenvironment.

The second aspect of the present invention provides a method for preparing the cartilage tissue engineering complex according to the first aspect of the present invention, comprising the steps of: inoculating the cartilage gel or cartilage sheet pieces according to the first aspect of the present invention into a porous frame structure, and performing in vitro chondrogenic culture, thereby obtaining the cartilage tissue engineering complex.

In another preferred embodiment, the cartilage gel is inoculated into the porous frame structure by a direct filling method.

In another preferred embodiment, the cartilage sheet pieces are inoculated into the porous frame structure by method of centrifugation.

In another preferred embodiment, no liquid is added to the centrifugal system of the method of centrifugation, and the cartilage sheet pieces are allowed to enter the frame structure by repeated centrifugation.

In another preferred embodiment, the chondrogenic culture is an in vitro culture using a chondrogenic medium.

In another preferred embodiment, the chondrogenic medium has the following components: high-glucose DMEM medium, serum substitute, proline, vitamin C, transforming growth factor-β1 (TGF-β1), insulin-like growth factor 1 (IGF-I) and dexamethasone.

In another preferred embodiment, the serum substitute is ITS premix, which comprises insulin, transferrin, selenite, linoleic acid, bovine serum albumin, pyruvate, ascorbic acid phosphate.

In another preferred embodiment, the time for chondrogenic culture is 3-15 days, preferably 5-11 days.

The third aspect of the present invention provides a use of the cartilage tissue engineering complex according to the first aspect of the present invention for preparing a medical product for repairing joint defects.

In another preferred embodiment, the joint defect is an articular cartilage defect.

In another preferred embodiment, the joint defect is a knee joint defect, an elbow joint defect, a hip joint defect, an ankle joint defect, a wrist joint defect, a mandibular joint defect, and a combination thereof.

The fourth aspect of the present invention provides a pharmaceutical composition, comprising the cartilage gel or cartilage sheet pieces according to the first aspect of the present invention and a pharmaceutically acceptable carrier.

In another preferred embodiment, the pharmaceutical composition is a liquid preparation.

In another preferred embodiment, the pharmaceutical composition is an injection.

In another preferred embodiment, the volume ratio of cartilage gel to pharmaceutically acceptable carrier in the pharmaceutical composition is 50% to 90%, preferably 70% to 85%.

In another preferred embodiment, the volume ratio of cartilage sheet pieces to pharmaceutically acceptable carrier in the pharmaceutical composition is 45% to 90%, preferably 60% to 80%.

The fifth aspect of the present invention provides a method for repairing joint defects, wherein the cartilage tissue engineering complex according to the first aspect of the present invention is used to transplant into the defective joint of a patient to be repaired.

In another preferred embodiment, the joint defect is an articular cartilage defect.

In another preferred embodiment, the joint defect is a knee joint defect, an elbow joint defect, a hip joint defect, an ankle joint defect, a wrist joint defect, a mandibular joint defect, and a combination thereof.

It should be understood that within the scope of the present invention, each technical features of the present invention described above and in the following (such as examples) may be combined with each other to form a new or preferred technical solution, which is not listed here due to space limitations.

### DESCRIPTION OF THE DRAWINGS

FIG. 1 shows an electron microscope photo of a decalcified bone matrix material. The bar in the figure is 1 mm.

FIG. 2 shows a general photo of the decalcified bone matrix material. The bar in the figure is 1 cm.

FIG. 3 shows schematic diagrams of ear cartilage gel and ear cartilage sheet obtained by culturing ear chondrocytes for 3 days and 15 days. Among them, A-C is ear cartilage gel cultured for 3 days, D-E is ear cartilage sheet cultured for 15 days (D and E), and ear cartilage sheet pieces formed by mincing (F).

FIG. 4 shows a physical diagram of the cartilage gel-frame structure complex. The bar in the figure is 1 cm.

FIG. 5 shows a physical diagram of the cartilage sheet pieces-frame structure complex. The bar in the figure is 1 cm.

FIG. 6 shows an electron microscope photo of the cartilage gel-decalcified bone matrix complex. The bar in the figure is 200 μM.

FIG. 7 shows an electron microscope photo of the cartilage sheet pieces-decalcified bone matrix complex. The bar in the figure is 200 μM.

FIG. 8 shows a comparison of the adhesion rate of the inoculated sample (cell suspension or cartilage gel) on the decalcified bone matrix after culture for 24 hours.

FIG. 9 shows a comparison of gel cartilage-decalcified bone complex (A) and simple decalcified bone (B) transplanted to the knee joint defect site in a goat.

### DETAILED DESCRIPTION

After extensive and in-depth research, the inventors surprisingly discovered and developed a cartilage tissue engineering complex for the first time. The cartilage tissue engineering complex is an integrated cartilage gel/cartilage sheet pieces-frame structure complex. Experiments have proved that by obtaining the primary cartilage for expansion culture, a specific number of chondrocytes are inoculated on and/or spread on a flat or basically flat culture surface, so that the inoculated chondrocytes form a specific laminated structure, and the laminated chondrocytes are cultured under suitable gelation culture conditions. A novel gel-like cartilage or sheet-like cartilage can be formed due to different culture times. Combining the prepared gel-like cartilage or sheet-like cartilage with the porous frame structure, the prepared cartilage tissue engineering complex can be regenerated into articular cartilage at the defective joint after being transplanted into the defective joint to repair and reconstruct the articular cartilage. On this basis, the present invention has been completed.

### Term

Unless otherwise defined, all technical and scientific terms used herein have the same meanings as those commonly understood by those skilled in the art to which the present invention belongs.

As used herein, the term “cartilage tissue engineering complex” includes the cartilage gel-frame complex and the cartilage sheet pieces-frame complex that have been chondrogenic cultured in vitro or have not been chondrogenic cultured in vitro as described herein. In the present invention, they can be uniformly referred to as cartilage tissue engineering complex.

As used herein, the term “inoculation” means the inoculation of chondrocytes in a cell culture dish, and may also mean the inoculation of cartilage gel/cartilage sheet pieces into a specified frame structure and the uniform distribution thereof. The meaning of “inoculation” as used can be understood by those skilled in the art from the context.

As used herein, when used in reference to specific enumerated values, the term “about” means that the value may vary by no more than 1% from the enumerated value. For example, as used herein, “about 100” includes all values between 99 and 101 (e.g., 99.1, 99.2, 99.3, 99.4, etc.).

As used herein, the term “contains” or “includes (comprises)” may be open, semi-closed and closed. In other words, the term also includes “substantially consisting of . . . ” or “consisting of . . . ”.

### Cartilage Gel and Preparation Thereof

As used herein, “gel cartilage”, “cartilage gel”, “gel-state cartilage”, “gel-like cartilage”, “cartilage gel of the present invention” or “gel cartilage of the present invention” can be used interchangeably, all refer to the cartilage (stem) cells in gel state of the present invention, in particular, chondrocytes with a specific concentration are inoculated on and/or spread on a flat or substantially flat culture surface, so that the inoculated chondrocytes form a laminated structure, and the chondrocytes having a laminated structure are cultured under suitable gelation culture conditions, thereby forming a gel-like cartilage culture.

The gel cartilage of the present invention is a new type of cartilage different from free chondrocytes, centrifugally precipitated chondrocytes and cartilage pellet. The gel cartilage of the present invention can be regarded as a specific form of cartilage between free chondrocytes and dense cartilage pellet. During the process of gelation culture of the gel cartilage of the present invention, the chondrocytes not only contact and/or interact with adjacent cells on the plane (X-Y plane), but also contact and/or interact with adjacent chondrocytes in multiple directions such as above and/or below and/or the upper or lower side, so as to promote the chondrocytes to secrete and form more extracellular matrix, therefore, the gelation cultured chondrocytes are wrapped in extracellular matrix with a certain viscosity, so that the gel cartilage of the present invention has a close connection, but has a certain viscosity and fluidity, so that the gel cartilage of the present invention is more suitable for inoculation and loading on various carrier materials (especially porous carrier materials), thereby forming a complex for repairing cartilage.

In addition, the gel cartilage of the present invention has a gel state on the one hand, and on the other hand, it has an unusually high cell density (usually at least 1.0×10⁸ cells/ml or more, such as 1.0×10⁸-10×10⁸ cells/ml). Therefore, it is particularly suitable for the preparation of grafts for repairing various types of cartilage, or for cartilage transplantation or cartilage repair surgery.

In the present invention, the complex for repairing cartilage includes the complex formed by loading the gel cartilage of the present invention on a carrier material (especially a porous biological frame structure) without undergoing chondrogenic culture, and also includes the complex formed by loading the gel cartilage of the present invention on a carrier material (especially a porous biological frame structure) and undergoing chondrogenic culture.

In the present invention, the complex suitable for transplantation to a human or animal body is the cartilage tissue engineering complex of the present invention, that is, the complex formed by loading the gel cartilage of the present invention on a carrier material (especially a porous biological frame structure) and undergoing chondrogenic culture.

Preferably, in the present invention, the gel cartilage is formed by in vitro culture for a period of time t1 under the gelation culture condition. Preferably, the t1 is 2.5-5.5 days, preferably 3-5 days.

In the present invention, one feature is laminated inoculation, that is, after chondrocytes with a specific density are inoculated into a culture container, the inoculated chondrocytes will form a multilayer chondrocyte group (i.e., a chondrocyte group with a laminated structure) through, for example, deposition. Typically, calculated on the basis of the culture area of the culture dish (or culture container), and assuming that the degree of confluence of the monolayer cells is 100%, the cell number S1 of the laminated inoculation of the present invention is n times of the cell number S0 for the of 100% confluence degree (i.e., S1/S0=n), wherein n is 1.5-20, preferably 2-10, more preferably 2.5-5.

### Cartilage Sheet and Preparation Thereof

As used herein, “cartilage sheet”, “sheet-like cartilage”, or “cartilage sheet of the present invention” can be used interchangeably, all refer to the cartilage (stem) cells in sheet state of the present invention, in particular, referring to the chondrocytes with a specific concentration are inoculated on and/or spread on a flat or substantially flat culture surface, so that the inoculated ear chondrocytes form a laminated structure, and the chondrocytes having a laminated structure are cultured under suitable culture conditions, thereby forming a sheet-like cartilage culture.

The “cartilage sheet” of the present invention is prepared on the basis of the preparation of the “cartilage gel” of the present invention by prolonging the gelation culture time. That is, in the present invention, the chondrocytes are inoculated and/or spread on a flat or substantially flat culture surface and cultured in vitro for a period of time t2 under gelation culture conditions, thereby forming a cartilage sheet. Preferably, the t2 is 6-30 days, preferably 7-20 days, and most preferably 10-15 days.

On the one hand, the cartilage sheet of the present invention has an unusually high cell density (usually at least 1.0×10⁸ cells/ml or more, such as 1.0×10⁸-10×10⁸ cells/ml). On the other hand, its thickness is thin (only 0.2-0.25 mm) and it has good toughness, and can be cut into “cartilage sheet pieces” with an average volume of 0.2 μ. They can be filled in the porous frame structure by simple centrifugation. Therefore, it is especially suitable for preparing and repairing various types of cartilage grafts, or for cartilage transplantation or cartilage repair surgery.

In the present invention, the complex for repairing cartilage includes the complex formed by loading the cartilage sheet pieces of the present invention on a carrier material (especially a porous frame structure) without undergoing chondrogenic culture, and also includes the complex formed by loading the cartilage sheet pieces of the present invention on a carrier material (especially a porous frame structure) and undergoing chondrogenic culture.

In the present invention, the complex suitable for transplantation to a human or animal body is the cartilage tissue engineering complex of the present invention, that is, the complex formed by loading the cartilage sheet pieces of the present invention on a carrier material (especially a porous frame structure) and undergoing chondrogenic culture.

As used herein, “specific concentration” or “specific density” refers to inoculating 1.0×10⁷-2.0×10⁷ cells, preferably 1.5×10⁷ cells, into a 3.5 cm culture dish (e.g., one well in a six-well plate). After gelation culture for different times, the cartilage gel containing chondrocytes with a density of 1.0×10⁸-10×10⁸ cells/ml or the cartilage sheet containing chondrocytes with a density of 1.0×10⁸-10×10⁸ cells/ml is finally formed.

In another preferred embodiment, the gelation culture condition is: inoculating chondrocytes with a specific density, and using gelation medium for culture, wherein the gelation medium is high-glucose (4-5 wt % glucose) DMEM medium containing 10% fetal bovine serum and 100 U/ml penicillin-streptomycin.

As used herein, the term “chondrogenic culture” refers to culturing a porous frame structure inoculated with cartilage gel or cartilage sheet pieces using chondrogenic medium to eventually form an integrated cartilage gel-frame structure complex or cartilage sheet pieces-frame structure complex, i.e., the cartilage tissue engineering complex of the present invention, for transplantation into cartilage defects in human or animal bodies.

### Cartilage and Chondrocytes

Cartilage refers to cartilage tissue, which is composed of chondrocytes and intercellular substance. The matrix in the cartilage is in gel state and has greater toughness. Cartilage is a connective tissue with main supporting function. There are no blood vessels and lymphatic vessels in cartilage. Nutrients infiltrate into the intercellular substance from the blood vessels in the perichondrium and then nourish bone cells.

Cartilage can be divided into 3 types according to the different intercellular substance, namely hyaline cartilage, elastic cartilage and fibrocartilage. The matrix of hyaline cartilage is composed of collagen fibers, fibrils and surrounding amorphous matrix. During the embryonic period, it has the function of temporary scaffold, which is later replaced by bone. In adults, hyaline cartilage is mainly distributed in the trachea and bronchial wall, the sternal end of the ribs and the surface of the bone (articular cartilage). In addition to collagen fibers, there are elastic fibers in the matrix of elastic cartilage. This kind of cartilage has greater elasticity and is mainly distributed in the auricle, external auditory canal wall, eustachian tube, epiglottis, and throat, etc. There are bundles of collagen fibers in the fibrocartilage matrix arranged in parallel or cross, which are tough. It is distributed in the intervertebral disc, glenoid, articular disc and some tendons, ligaments, to enhance the flexibility of movement and protection, support and other functions.

The chondrocytes used in the cartilage tissue engineering complex of the present invention can be hyaline cartilage cells, elastic cartilage cells or fibrocartilage cells taken from hyaline cartilage, elastic cartilage or fibrocartilage, which can be transformed into articular cartilage in the joint environment after being implanted into the joint defect of the subject.

### MACI and ACI

MACI is the abbreviation of matrix-induced autologous chondrocyte implantation, which means “matrix-induced autologous chondrocyte transplantation”. It is a technology that uses tissue engineering technology to carry out chondrocyte transplantation. MACI is the latest and best technology for the treatment of articular cartilage defects in the world.

ACI is the abbreviation of autologous chondrocyte implantation, which means “autologous chondrocyte transplantation”. It is one of the widely used tissue engineering techniques for the treatment of articular cartilage defects. After the advent of MACI, ACI was called “traditional ACT” accordingly to distinguish it from MACI.

### Porous Frame Structure

As used herein, the term “porous frame structure” refers to a carrier made of a biocompatible material having a certain number of pores on its surface and interior to facilitate the attachment of the ear cartilage gel or ear cartilage sheet inoculated thereon. In the present invention, the biocompatible material is preferably a biodegradable material.

A biodegradable material refers to a material that can be decomposed in the body after being transplanted into an animal. The porous frame structure of the ear cartilage tissue engineering graft of the present invention is made of a biodegradable material selected from the group consisting of PCL, PGA, allogeneic bone repair material, xenogeneic bone repair material, decalcified bone matrix, and a combination thereof, but not limited to the materials described above. Among them, allogeneic bone repair materials and xenogeneic bone repair materials include decalcified bone matrix materials. In a preferred embodiment of the present invention, the porous frame structure of the ear cartilage tissue engineering complex is a decalcified bone matrix.

### Decalcified Bone Matrix

The decalcified bone matrix used in the preferred embodiment of the present invention has a thickness of 0.3-0.8 cm, preferably 0.4-0.6 cm, and most preferably 0.5 cm. The decalcification amount of the decalcified bone matrix is 30% to 50%, the degree of decalcification of which is appropriate, and the supporting effect is good, and the decalcified bone matrix is easy to trim and cut to a suitable shape and size. The pore size of the demineralized bone matrix is 400-800 μm, which is easy to be filled with chondrocytes.

Decalcified bone matrix (DBM) is a bone graft material that can reduce immunogenicity by decalcification of allogeneic bone or xenogeneic bone. Different degrees of decalcification correspond to different mechanical strengths. It has good biological characteristics, osteoinductivity, osteoconductivity and biodegradability, which promotes new bone formation and bone tissue mineralization, and then accelerates bone healing. It can effectively repair bone damage alone or in combination with autologous bone, other biological materials and growth factors. It is an ideal bone tissue engineering scaffold material. However, the general decalcified bone matrix has a large pore size and extremely low cell adhesion rate when inoculated with chondrocyte suspension, which is not conducive to the construction of tissue engineering carriers.

In another preferred embodiment, the decalcified bone matrix of the present invention has a pore size of 400-800 μm and a porosity of 87.3%±3.7%.

### Allogeneic Bone Repair Material

Allogeneic bone is currently the most commonly used bone implantation material in orthopedics. It is mainly used to repair and fill bone defects and play a role of fixation and support. Allogeneic bone is taken from donated human bone tissue. “Allogeneic” indicates that it is from the human body but not from the patient's own body. After the donor is selected, it is usually obtained under sterile conditions within 24 hours of death, and processed immediately. Preservation methods include fresh freezing and freeze drying. Fresh frozen bone can be stored for 1 year at −20° C.; freeze dried bone can be stored at room temperature for a long time after vacuum packaging, and the antigenicity of that is lower. Compared with fresh frozen bone, the mechanical properties of freeze dried bone will be reduced by 50%, and ethylene oxide or high-dose γ-ray irradiation disinfection will further reduce the bone induction performance.

### Xenogenic Bone Repair Material

Xenogenic bone is a bone repair material derived from other species such as cattle or pig, etc. It has a wide range of sources and relatively low prices. However, the immunogenicity of xenogeneic bone is strong, and it is easy to cause immune rejection after implantation in patients. In addition, xenogeneic bone has no ability to induce the proliferation of mesenchymal stem cells, and its biological activity is poor. It needs to be combined with other repair materials or related cytokines to achieve the repair effect.

### The Medium Used in the Present Invention

Chondrogenic medium: high glucose DMEM medium, 1% 1×ITS premium (ITS universal culture mixture, containing insulin, transferrin, selenite, linoleic acid, bovine serum protein, pyruvate, ascorbic acid phosphate), 40 μg/ml proline, 10 ng/ml TGF-β 1, 100 ng/ml IGF-1, 40 ng/ml dexamethasone and 50 μg/ml vitamin C.

Gelation medium: DMEM medium containing 4-5 wt % glucose, 10% FBS (v/v) and 100 U/ml penicillin-streptomycin.

### Adhesion Rate

In the present invention, when the cartilage gel of the present invention is inoculated into a carrier material (especially a porous biological frame structure), the cartilage gel of the present invention has a certain adhesion rate, which is determined by the adhesion rate measurement method provided by the present invention. The adhesion rate of the cartilage gel of the present invention is ≥90%, preferably ≥95%.

The adhesion rate in the present invention is defined as follows:
Detect the DNA quantification Al of inoculated samples (e.g., cartilage gel); Detect the DNA quantification A2 of the post-inoculation complex (e.g., ear cartilage gel-frame complex) after culture for 24 hours. The adhesion rate is A2/A1*100%.

The method of determining the adhesion rate includes the following steps:
Taking inoculated samples (such as cartilage gel or cartilage gel-frame complex) and digesting them with protease K. The digested samples are quantitatively detected using PicoGreen kits (Invitrogen, Carlsbad, CA, USA), absorbance of 520 nm is determined using fluorescent microplate reader, and DNA content is calculated according to standard curve formula.

The main advantages of the present invention include:
(1) The cartilage gel of the present invention is more mature than chondrocytes and has certain fluidity.
(2) Decalcified bone matrix, as a biodegradable natural material, can be degraded in the body, which has a low body immune response and good biological safety.
(3) The decalcified bone matrix material has a large pore size and good porosity, but the cell adhesion rate is extremely low when inoculated with chondrocyte suspension. The use of cartilage gel-like tissue with certain fluidity and viscosity can effectively improve the adhesion rate.
(4) Simple injectable cartilage cannot be molded. Under tension conditions, the absorption rate of simple injectable cartilage alone is high, so that clinical application of which is limited. Using decalcified bone matrix as a frame structure, a special shape injectable cartilage decalcified bone matrix complex can be constructed, and the absorption rate of cartilage can be limited after providing mechanical support.
(5) The graft of the present invention has diverse cell sources and good amplification ability.
(6) Compared with other tissue engineering repair methods, cartilage gel/cartilage sheet pieces-decalcified bone matrix complex can regenerate cartilage stably.
(7) Cartilage gel/cartilage sheet pieces-decalcified bone matrix complex can regenerate cartilage stably and provide real-time mechanical support. The real-time repair effect of that is good.

Hereinafter, the present invention will be further described with reference to specific examples. The present invention is further explained below in conjunction with specific example. It should be understood that these examples are only for illustrating the present invention and not intend to limit the scope of the present invention. The experimental method without specific conditions in the present embodiment is generally in accordance with conventional conditions, or in accordance with the conditions recommended by the commodity manufacturer. Unless otherwise stated, percentages and parts are calculated by weight. Unless otherwise stated, the materials and reagents used in the examples of the present invention are commercially available products.

### EXAMPLE 1

### Preparation of Cartilage Gel-Decalcified Bone Complex

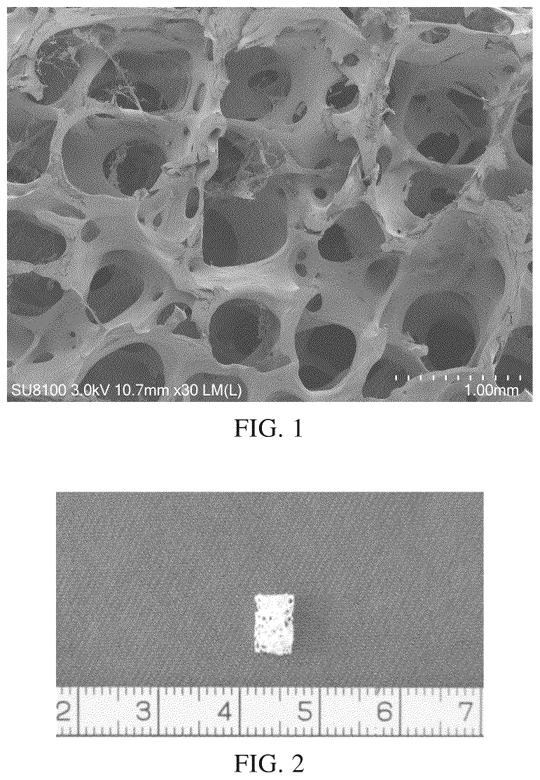

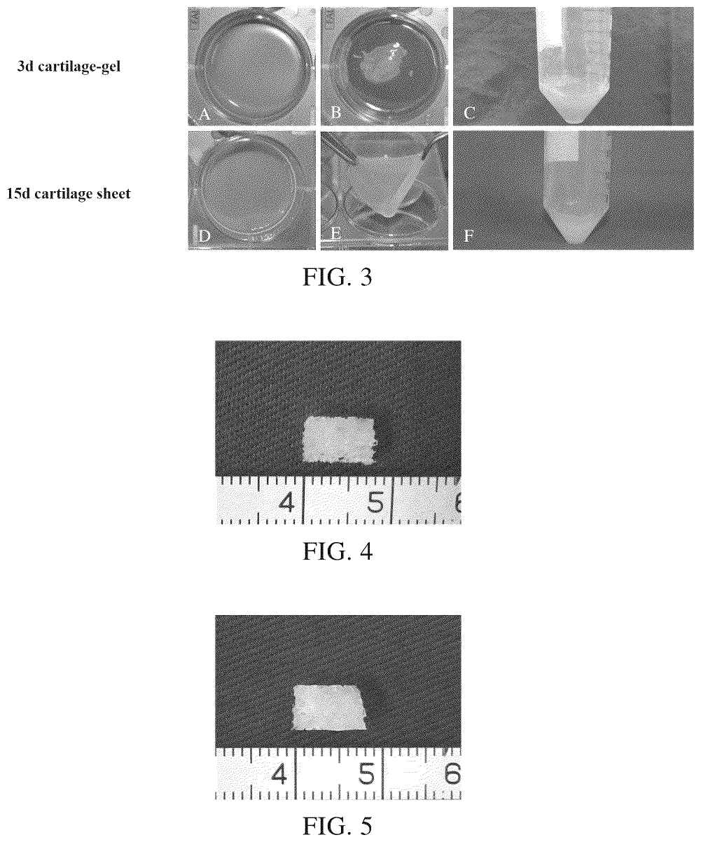

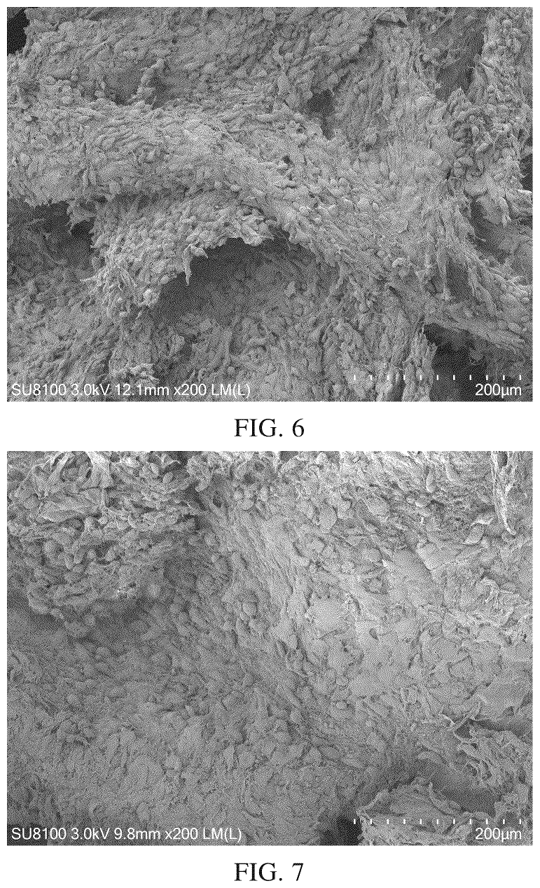

In this example, the cartilage gel-frame complex structure was prepared. The specific operation method is as follows:
(1) Part of the subject's autologous ear cartilage tissue was taken, and 2.5×2.5 cm² cartilage tissue was aseptically cut. The mucosa and fibrous tissue was peeled off from the cartilage surface by sterile instruments.
(2) The cartilage tissue was cut into 1.5×1.5 mm² cartilage pieces; 0.15% collagenase was prepared; and the cartilage pieces were added to the prepared collagenase for digestion for 8 hours.
(3) After 8 hours, collagenase solution was filtered and centrifuged to obtain ear chondrocytes, and primary and subculture were conducted using high-glucose DMEM medium containing 10% FBS;
(4) After expansion, the cells were collected and resuspended, and the cells were inoculated into a six-well plate with the cell volume of 8×10⁶ cells/10 ml to 30×10⁶ cells/10 ml/well, and were cultured in gelation medium (DMEM medium containing 4-5 wt % glucose, 10% FBS (v/v) and 100 U/ml penicillin-streptomycin).
(5) After 72 hours (3 days) of inoculation, the medium in the six-well plate was sucked off, and the gel-like cartilage tissue at the bottom of the six-well plate can be seen (A of FIG. 3). The gel-like cartilage at the bottom of the six-well plate was gathered by using a tweezer (B of FIG. 3). The gel cartilage yield of one well was 0.1-0.2 ml and collected into a 5 ml syringe. In the gel cartilage, the cell density was 1.0×10⁸ cells/ml-10×10⁸ cells/ml, preferably 1.5-5×10¹⁰ cells/ml. It was mixed with 0.15 ml of medium to make a preparation containing cartilage gel, as shown in C of FIG. 3.
(6) The cartilage gel preparation (prepared in step (5), volume was about 0.25-0.35 ml) was inoculated in the frame structure (decalcified bone matrix frame (FIG. 1 and FIG. 2, with a pore size of about 400-800 μm, and a porosity of about 87.3%±3.7%), and placed at 37° C., 95% humidity, 5% CO₂ for 2 hours.
(7) Then chondrogenic medium was added to continue in vitro culture for 3-11 days to form the cartilage gel-frame structure complex, as shown in FIG. 4. Electron microscopy observation shows that the pores of the decalcified bone matrix are filled with chondrocytes (FIG. 6).

When it is used for joint defect repair, the shape and size of the cartilage to be repaired can be determined according to the early MRI, CT and other auxiliary examinations, then the cartilage gel frame structure complex can be cut.

### EXAMPLE 2

### Preparation of Cartilage Sheet Pieces-Decalcified Bone Complex

In this example, the cartilage sheet pieces-frame complex was prepared. The specific operation method is as follows:
(1) 2.5×2.5 cm² ear cartilage tissue was aseptically cut, and the mucosa and fibrous tissue was peeled off from the cartilage surface by sterile instruments.
(2) The cartilage tissue was cut into 1.5×1.5 mm² cartilage pieces; 0.15% collagenase was prepared; and the cartilage pieces were added to the prepared collagenase for digestion for 8 hours.
(3) After 8 hours, collagenase solution was filtered and centrifuged to obtain ear chondrocytes, and primary and subculture were conducted.
(4) After expansion, the cells were collected and resuspended, and the cells were inoculated into a six-well plate with the cell volume of 8×10⁶ cells/10 ml to 30×10⁶ cells/10 ml/well, and cultured in gelation medium (DMEM medium containing 4-5 wt % glucose, 10% FBS (v/v) and 100 U/ml penicillin-streptomycin). After 24 hours or 48 hours of culture, the fresh gel culture medium was replaced and in vitro culture was continued until Day 15.
(6) The culture medium in the six-well plate was sucked off, and the cartilage membrane tissue at the bottom of the six-well plate can be seen (D of FIG. 3), where the cell density in the cartilage membrane tissue was about 1.0×10⁸ cells/ml-10×10⁸ cells/ml.

The cartilage sheet was clamped with tweezers (E of FIG. 3) and cut into cartilage sheet pieces with a size of 1×1 mm², then the cartilage sheet pieces were collected into a 50 ml centrifuge tube, as shown in F of FIG. 3.
(7) The frame material to be inoculated (decalcified bone matrix frame (FIG. 4 and FIG. 5)) was placed in the centrifuge tube with cartilage sheet pieces to ensure that the frame material is completely immersed. The centrifuge tube containing frame material and cartilage sheet pieces was put into the centrifuge for centrifugation for 2 minutes at 600 rpm/min.
(8) The inoculated frame material was placed at 37° C., 95% humidity, and 5% carbon dioxide for a certain period of time; then chondrogenic medium was added to continue in vitro culture for 3-11 days to form the cartilage sheet pieces-frame structure complex, as shown in FIG. 5. Electron microscopy observation shows that the pores of the decalcified bone matrix are filled with chondrocytes (FIG. 7).

When it is used for joint defect repair, the shape and size of the cartilage to be repaired can be determined according to the early MRI, CT and other auxiliary examinations, then the cartilage sheet pieces-frame structure complex can be cut.

### Comparative Example 1

### Determination of Adhesion Rate of Chondrocyte Suspension and Cartilage Gel

A decalcified bone matrix framework was provided (as shown in FIG. 2). The gel cartilage preparation (prepared in Example 1, with a volume of about 0.25-0.35 ml) was inoculated into the above decalcified bone matrix framework. The DNA content of the gel cartilage preparation was measured before inoculation.

Primary cultured chondrocytes were subcultured 4 times at 37° C., 95% humidity, and 5% carbon dioxide, and then the cell culture medium was added to prepare a cell suspension. The DNA content of the prepared chondrocyte suspension was measured. The cell suspension was inoculated into the aforementioned decalcified bone matrix framework.

The inoculated cartilage gel-decalcified bone matrix complex and chondrocyte suspension-decalcified bone matrix complex were placed in an incubator at 37° C., 95% humidity and 5% carbon dioxide for 24 hours. The two types of complexes were sampled to measure the DNA content of each, respectively.

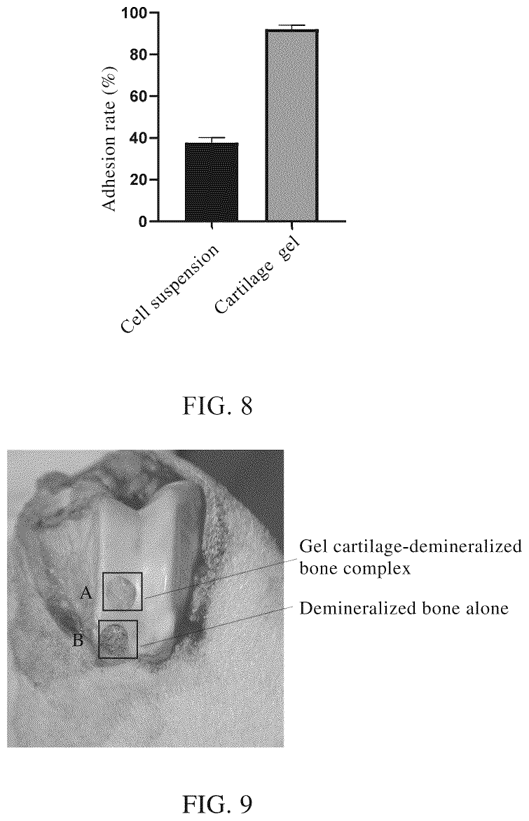

As shown in FIG. 8, the adhesion rate is calculated using the adhesion rate measurement method described in the present specification. Compared with the cell suspension, the adhesion rate of the gel cartilage of the invention is 92%±2%, which is about 3-fold of the adhesion rate of the cell suspension.

### Comparative Example 2

### Animal Transplantation Experiment for Repairing Articular Cartilage

A cartilage defect with a diameter of 7.5 mm was made on the articular surface of the knee joint of the experimental animal, and the gel cartilage-frame complex (gel cartilage-decalcified bone complex) prepared in Example 1 and pure decalcified bone matrix were implanted into defect position A and defect position B, respectively.

The repair of cartilage defects in the animal body after implantation was immediately observed.

The results are shown in FIG. 9.

The defect area at position A is smooth and solid, surrounded by a soft tissue membrane, with a certain degree of elasticity and excellent immediate repair effect.

The wound at the defect B is rough and has only physical support, which cannot be repaired immediately.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A cartilage tissue engineering complex, which comprises:
(a) a carrier comprising a porous frame structure; and
(b) cartilage gel or cartilage sheet pieces containing chondrocytes that are inoculated on or loaded on the carrier.

2. The complex of claim 1, wherein the chondrocytes comprise elastic cartilage cells, fibrocartilage cells, hyaline cartilage cells and a combination thereof.

3. The complex of claim 1, wherein the cartilage gel comprises a cell population of chondrocytes and extracellular matrix secreted by chondrocytes, wherein the extracellular matrix encapsulates the cell population, and the cartilage gel is in a gel state, and the density of chondrocytes is at least 1.0×10⁸ cells/ml or 1.0×10⁸ cells/g.

4. The complex of claim 3, wherein the adhesion rate of the cartilage gel is ≥90%, preferably ≥95%.

5. The complex of claim 3, wherein the cartilage gel is obtained by gelation culture for 2.5-5.5 days, preferably for 3-5 days.

6. The complex of claim 1, wherein the cartilage sheet pieces comprise a cell population composed of chondrocytes and extracellular matrix secreted by chondrocytes, wherein the extracellular matrix encapsulates the cell population, and the cartilage pieces are prepared by mincing the cartilage sheet, wherein the density of chondrocytes is at least 1.0×10⁸ cells/ml or 1.0×10⁸ cells/g.

7. The complex of claim 6, wherein the cartilage sheet is obtained by gelation culture for 6-30 days, preferably 7-20 days, and most preferably 10-15 days.

8. The complex of claim 5, wherein the gelation medium contains the following components: high-glucose DMEM medium containing 4 to 5 wt % glucose, 10% FBS (v/v) and 100 U/ml penicillin-streptomycin.

9. The complex of claim 1, wherein the porous frame structure is made of a biodegradable material selected from the group consisting of PCL, PGA, allogeneic bone repair material, xenogeneic bone repair material, or decalcified bone matrix.

10. The complex of claim 9, wherein the porous frame structure is a decalcified bone matrix, with a thickness of 0.3-0.8 cm, preferably 0.4-0.6 cm, and most preferably 0.5 cm, and the decalcified amount of the decalcified bone matrix is 30-50%.

11. A method for preparing the cartilage tissue engineering complex of claim 1, comprising the steps of: inoculating the cartilage gel or cartilage sheet pieces into a porous frame structure, and after performing in vitro chondrogenic culture, the cartilage tissue engineering complex is obtained.

12. The method of claim 11, wherein the chondrogenic culture is in vitro culture using chondrogenic medium, and the chondrogenic medium has the following components: high-glucose DMEM medium, serum substitute, proline, vitamin C, transforming growth factor-β1 (TGF-β1), insulin-like growth factor 1 (IGF-I) and dexamethasone.

13. Use of the cartilage tissue engineering complex of claim 1 for preparing a medical product for repairing joint defects.

14. A pharmaceutical composition comprising the cartilage gel or cartilage sheet pieces of claim 1 and a pharmaceutically acceptable carrier.

15. A method for repairing joint defects, wherein the cartilage tissue engineering complex of claim 1 is used to transplant into the defective joint of a patient to be repaired.

16. The complex of claim 7, wherein the gelation medium contains the following components: high-glucose DMEM medium containing 4 to 5 wt % glucose, 10% FBS (v/v) and 100 U/ml penicillin-streptomycin.

17. The method of claim 11, wherein the cartilage gel comprises a cell population of chondrocytes and extracellular matrix secreted by chondrocytes, wherein the extracellular matrix encapsulates the cell population, and the cartilage gel is in a gel state, and the density of chondrocytes is at least 1.0×10⁸ cells/ml or 1.0×10⁸ cells/g.

18. The method of claim 11, wherein the cartilage sheet pieces comprise a cell population composed of chondrocytes and extracellular matrix secreted by chondrocytes, wherein the extracellular matrix encapsulates the cell population, and the cartilage pieces are prepared by mincing the cartilage sheet, wherein the density of chondrocytes is at least 1.0×10⁸ cells/ml or 1.0×10⁸ cells/g.
